(19) United States
(12) Patent Application Publication
(10) Pub. No.: US 2026/0028307 A1
(43) Pub. Date: Jan. 29, 2026

(54) Process for the hydrogenation of MDA

(71) Applicant: Evonik Operations GmbH, Essen (DE)

(72) Inventors: Florian BOECK, Witten (DE); Sven SCHNEIDER, Datteln (DE); Martina LUDWIG, Recklinghausen (DE)

(73) Assignee: Evonik Operations GmbH, Essen (DE)

(21) Appl. No.: 19/283,076

(22) Filed: Jul. 28, 2025

(30) Foreign Application Priority Data
Jul. 26, 2024 (EP) 24191090.0

(51) Int. Cl. C07C 209/72; B01J 23/46

(52) U.S. Cl. CPC C07C 209/72; B01J 23/462

(57) ABSTRACT

A process for continuous heterogeneous catalytic hydrogenation of MDA. The process occurs in a reactor cascade including n serially connected reaction spaces, Rᵢ, that are each filled with catalyst and can be filled or emptied independently of one another where 1≤i≤n, in the order in which they are connected. R₁ is temporarily disconnected from the cascade as soon as the catalyst present in R₁ has in the course of the reaction become deactivated to an undesirable degree. The reconfigured reactor cascade R_{i′}, includes i′ reaction spaces where 1≤i′≤(n−1), in the order in which they are connected. Each reaction space Rᵢ where 2≤i≤n becomes a reaction space R_{i′} where 1≤i′≤(n−1). The catalyst in R₁ is replaced and/or regenerated and R₁ is subsequently connected as reaction space R_{i′} where i′=n.

The present invention relates to a process for the hydrogenation of MDA.

Methylenebis(cyclohexylamine) is an important industrial chemical that is used in many typical amine reactions, such as reactions with carboxylic acids, phosgene, aldehydes, ketones and epoxides. Methylenebis(cyclohexylamine) allows use to be made of the advantages of cycloaliphatic amines in epoxy systems: low mixed viscosities, moderate reactivity and little exothermic behaviour, as well as outstanding mechanical properties and excellent chemical resistance. By comparison with other amines, the tendency to carbamate formation is reduced, which is an advantage for use as an epoxy curing agent.

Methylenebis(cyclohexylamine) is a cycloaliphatic amine that is solid or liquid under standard conditions (SATP) and is typically produced by liquid-phase hydrogenation of MDA. The acronym MDA was introduced historically as an abbreviation for the product mixture that is formed in the reaction of aniline and formaldehyde, comprising mainly “methylenedianiline” (diaminodiphenylmethane), and is still used to describe the process product, which is now produced on an industrial scale. The hydrogenation product, comprising mainly methylenebis (cyclohexylamine), is therefore often also referred to as H12MDA.

As a consequence of the process by which it is produced, MDA is typically a mixture of different diaminodiphenylmethanes. It consists mainly of 4,4′-diaminodiphenylmethane. However, 2,4′ and 2,2′ isomers may also be present. MDA may also contain reaction products having three or more aromatic rings that are formed in the reaction of aniline and formaldehyde, in particular ones having three or more phenyl rings. These reaction products having three or more aromatic rings are also referred to as multiring compounds.

Because of the high proportion of 4,4′-diaminodiphenylmethane in the MDA used, commercially available methylenebis(cyclohexylamine) is mostly 4,4′-diaminodicyclohexylmethane, or bis(para-aminocyclohexyl)methane. The potential presence of the corresponding 2,4′- and 2,2′-diaminophenylmethane isomers in the MDA means that 2,4′-diaminodicyclohexylmethane and 2,2′-diaminodicyclohexylmethane may likewise be present in methylenebis(cyclohexylamine). In addition, hydrogenated MDA can also contain (sometimes partially) hydrogenated multiring compounds besides methylenebis(cyclohexylamine).

U.S. Pat. No. 5,578,546 A discloses that a process for producing methylenebis(cyclohexylamine) was first described in 1947 and converted to industrial scale in 1965.

The hydrogenation of MDA is highly exothermic. For instance, WO 2010/069484 A1 reports a reaction enthalpy of −1600 kJ/mol.

Hydrogenation results in the formation of various diastereoisomers, depending on the process. The 4,4′-diaminodicyclohexylmethane product derived from 4,4′-diaminodiphenylmethane can be in the form of trans/trans, cis/cis and cis/trans isomer and is therefore generally a mixture of these isomers in varying proportions. The melting point of the compound rises with increasing trans/trans content. Fields of use accordingly vary significantly depending on the isomer content: Whereas methylenebis(cyclohexylamine) grades having a low trans/trans content (for example 10-30% by weight) are used in the field of amine and isocyanate crosslinkers, especially in the field of two-component resins, grades having a high trans/trans content (for example ≥48% by weight) are mainly used as regulator in polyamide compounds. The production of products having a low trans/trans content represents a particular challenge, since the thermodynamic equilibrium, as described in U.S. Pat. No. 3,636, 108 A, is in the range of significantly higher trans/trans contents (up to 51.2%). Moreover, U.S. Pat. No. 2,606,925 A shows that prolonged heating can subsequently shift the equilibrium towards a higher proportion of trans/trans isomer.

The composition of the hydrogenation product also depends on the composition of the MDA used: MDA is commonly used in grades from MDA50 to MDA100, with the number between 50 and 100 indicating the content of diaminodiphenylmethanes in the MDA mixture. MDA50 is an MDA grade that, as explained above, for process-related reasons contains about 50% by weight of diaminodiphenylmethanes and 50% by weight of multiring compounds. The individual multiring compounds can be referred to as 3-ring compounds, 4-ring compounds, etc., according to the number of aromatic rings present. MDA50 is the grade produced in the highest volume and is mainly processed to methylenedicyclohexyl diisocyanate (MDI). MDA100 is pure MDA, or diaminodiphenylmethane without multiring compounds. MDA85 and MDA90 are other grades of medium purity available on the market. When patent specifications relating to processes for producing methylenebis(cyclohexylamine) mention the purity of the MDA grade, the grade concerned is usually MDA100 (for example CN 110204447 B). US 2005/261525 A1 by contrast focuses on the hydrogenation of MDA50. The hydrogenated oligomeric amines obtained here as high boilers are suitable as crosslinkers having particularly low vapour pressures for a range of specialty applications, as demonstrated in US 2004/162409 A1.

The content of (sometimes partially) hydrogenated multiring compounds in the product decreases in the order of the reactants MDA50, MDA85, MDA90, MDA100, since the content of multiring compounds decreases from MDA50 to MDA100.

There are descriptions in the literature of hydrogenations of MDA with catalysts containing active metals selected from cobalt (for example U.S. Pat. No. 3,743,677 A), nickel (for example U.S. Pat. No. 4,503,251 A), ruthenium (for example U.S. Pat. Nos. 2,494,563 A, 2,606,925 A, 2,606,928 A, 3,959,374 A, 3,636,108 A and 4,161,492 A), rhodium (for example DE 24 23 639 C3) and iridium (for example U.S. Pat. No. 3,914,307 A) or combinations thereof.

For obtaining product having low trans/trans content alongside high conversions and good selectivities, catalysts based on ruthenium and rhodium have in particular become established. These catalysts are employed in more recent patent specifications primarily in the form of supported catalysts. For instance, FR 2372142 A1 discloses a catalyst on aluminium oxide, while US 2016/304436 A1 employs zirconium oxide as support. The support, BET value of the support and the pore size have many times been described as having a critical influence on the lifetime of the catalyst (for example U.S. Pat. No. 5,773,657 A, CN 102008969 B, US 2002/087036 A1, US 2004/034252 A1). In some cases basic supports have been deliberately used. For instance, U.S. Pat. No. 5,578,546 A1 describes a catalyst on basic alumina, while U.S. Pat. No. 6,184,416 B1 discloses lithium aluminium oxide as basic support.

Many of the literature sources cited above refer to the short lifetime of the catalyst as the biggest weak point of the process. U.S. Pat. No. 3,636, 108 A reports a steady fall in the activity of the catalyst due to coking or blocking of the active surface by oligomeric compounds. It is disclosed that this deactivation process can be reduced by adding ammonia and alkali metal. Ammonia in combination with an aliphatic alcohol as additive is also described in U.S. Pat. No. 5,214,212 A.

The use of alkali metal and/or alkaline earth metal as promoters to boost activity, selectivity, and/or lifetime is also demonstrated by other patent specifications: U.S. Pat. No. 3,697,449 A discloses the beneficial effect on the hydrogenation of MDA and other aromatic amines of adding alkali metal hydroxides or alkoxides as in-situ promoters. JP 2002/348267 A1 discloses the addition of Ca(OH)₂ as basic moderator for avoiding laborious catalyst regeneration processes. U.S. Pat. No. 6,184,416 B1 demonstrates the higher activity of a rhodium catalyst applied directly to a lithium aluminium oxide support. JP H08-092175 A describes a system in which alkali metal carbonate in diethylene glycol dimethyl ether as solvent is added to the noble metal catalyst. U.S. Pat. No. 6,075, 167 A shows that adding a metal nitrite makes it possible to shorten the reaction time and reduce the formation of by-products. U.S. Pat. No. 4,448,995 A employs nitrates or sulfates of alkali metals or alkaline earth metals as moderators. U.S. Pat. No. 4, 186,145 A discloses combining organic and/or organic alkali metal compounds together with oxides, hydrated oxides or hydroxides of chromium and manganese as promoters. CN 111804324 B discloses that adding lithium amide makes it possible to reduce oligomerization of methylenebis(cyclohexylamine) and aminoalcohol formation. A reported advantage over other lithium salts is its greater solubility in an organic solvent. Lastly, CN 116023272 A describes the use of lithium formate, acetate or oxalate as promoter to increase throughput and to control the trans/trans content in the stirred-tank reactor cascade.

All the described additions of alkali metal salts have the disadvantage that these accumulate in the high boilers later on in the process, which as a consequence can no longer be used as a product of value. Moreover, the described additives have the disadvantage of corroding the reactor.

Organic additives for extending the lifetime have likewise been repeatedly described (for example CN 106631826 B, CN 103265438 B or CN 115870013 A). A disadvantage here too is that these products remain as impurities in the process.

A further variant for extending the lifetime is disclosed in US 2010/292510 A1. Here, inorganic additives such as aluminium oxide or silicon oxide are added to the reaction to bind catalyst poisons. In U.S. Pat. No. 6,998,507 B1, MDA in the presence of hydrogen is partially hydrogenated in the presence of a ruthenium catalyst and then reacted to completion in the presence of a rhodium catalyst. In this process the ruthenium catalyst is likewise used as an adsorbent for catalyst poisons. The two-step reaction regimen does however make the process more complicated.

As a general rule, with promoters or moderators too, the lifetime of the employed catalysts is severely limited by comparison with other processes. Oligomers introduced from the MDA itself or from the hydrogenation process gradually coat the surface of the catalysts, resulting in their inhibition. In suspension processes, further catalyst is often added over time for maintenance. For a continuous fixed-bed process, this is not possible. A number of patent specifications have accordingly described special regeneration processes for the catalysts employed.

For instance, CN 117654548 A describes the regeneration of the catalyst for the production of methylenebis(cyclohexylamine) from MDA by hot treatment with a long-chain alcohol followed by washing with a polar solvent and activation in a stream of hydrogen.

CN 110204447 B describes a regeneration process for a catalyst used for the hydrogenation of MDA in which the catalyst is inter alia washed multiple times with alkali-metal-containing ammonia and then heated to high temperature.

CN 113893866 B discloses that the activity of the catalyst can be regained by washing with acid and water in an inert atmosphere followed by reactivation.

Regeneration processes are however disadvantageous on account of the associated costs.

Technical details both of hydrogenations of MDA with suspended catalysts and hydrogenations in fixed-bed processes have been described. Both processes have their advantages and disadvantages.

For instance, in a stirred-tank reactor, although the utilization rate of the suspended catalyst is very high, the product is in this case also exposed to high temperatures for very long periods and tends to isomerize to a trans/trans-enriched product. Moreover, stirred-tank reactors are limited in respect of mass transport from the gas phase to the solid catalyst. With stirred-tank reactors there is also the risk not just that reaction heat can be dissipated only poorly, but that the reaction, once started, cannot be paused.

CN 117623940 A describes that it is possible to obtain a methylenebis(cyclohexylamine) product having a low trans/trans isomer content in high conversion in a stirred-tank reactor cascade even with protracted use of the suspended catalyst, if the feed volume is reduced when conversion falls.

EP 231 788 B1 describes a process for the hydrogenation of MDA in the presence of a rhodium-containing and ruthenium-containing suspension catalyst. It is additionally disclosed that it is possible to obtain a product having a particularly low trans/trans content in high conversions at a relatively low hydrogen pressure of about 50 bar. Very high lifetimes and a low tendency to oligomerization are at the same time achieved. A disadvantage of the process is that the use of rhodium requires very high temperatures.

A disadvantage of suspension catalysts in general is that their removal, particularly in continuous processes, is technically laborious.

For instance, EP 1 251 119 A2 describes the production of diaminodicyclohexylmethane having a low trans/trans content in a continuously operated suspensions reactor. The reaction is preferably carried out in a stirred-tank reactor cascade. The disadvantage of this is that performance of the hydrogenation is possible only with very high technical complexity in order for the MDA to be completely hydrogenated.

EP 2 285 481 A1 discloses a combination of loop reactor and bubble column for optimal control of the reaction. The purpose of the loop reactor is to effectively remove reaction heat from the process. However, a loop reactor has limitations in respect of mass transport in particular.

In order to achieve advantageous properties, it is also possible for MDA to be hydrogenated in fixed-bed reactors. In particular, fixed-bed reactors designed as trickle-bed reactors have the advantage of permitting very good mass transport and thus generating smaller amounts of by-products. In addition, the reacted product remains in contact with the catalyst for the shortest-possible time, which likewise reduces side reactions, in this case isomerization. Tube-bundle reactors in addition have the advantage of being able to cool the reaction medium extremely efficiently. However, a disadvantage of continuously-operated fixed-bed reactors is that production needs to be paused when the activity of the catalyst declines and the catalyst needs to be replaced or reprocessed.

CN 116621711 B describes a continuous process for the production of 4,4′-diaminodicyclohexylmethane in which the hydrogenation is carried out in a fixed-bed reactor and the reaction is paused and subjected to distillative workup once the residual MDA content is 0-10%. The incompletely hydrogenated components are either hydrogenated further or returned to the process. This makes it possible to maintain the desired low trans/trans-methylenebis(cyclohexylamine) content despite the exposure to high temperatures. A disadvantage of this process is that production needs to be paused when the activity of the catalyst declines and the catalyst needs to be replaced or reprocessed.

WO 2010/069484 A1 discloses a process for producing bis (para-aminocyclohexyl) methane from methylenedianiline in which the reaction is carried out under adiabatic conditions in 5 to 50 serially-connected reaction zones in which the heterogeneous catalysts are present. Preference is given to using fixed-bed catalysts. Experimental data for this are not provided. However, the large number of interconnected reactors and the adiabatic reaction regimen are too complex for large-scale industrial use and make this process less economically attractive. In addition, a disadvantage of this process too is that production needs to be paused when the activity of the catalyst declines and the catalyst needs to be replaced or reprocessed.

WO 2009/144148 A1 discloses inter alia the hydrogenation of aromatic diamines to cycloaliphatic diamines and in particular the hydrogenation of MDA to methylenedicyclohexyldiamine. A preferred embodiment discloses the hydrogenation of aromatic amines and of MDA in particular in fixed-bed reactors. It is disclosed that it was found to be advantageous to carry out the reaction in two or more reaction spaces connected in series, since these can be temperature-controlled independently of one another and a partial replacement of catalyst is also possible. A disadvantage of this process too is that production needs to be paused when the activity of the catalyst declines and the catalyst needs to be replaced or reprocessed.

WO 2015/086638 A1 discloses a process for the hydrogenation of 4,4′-methylenedianiline and/or polymeric MDA with hydrogen in the presence of a zirconium-oxide-supported ruthenium catalyst. The reaction can be carried out continuously and in a fixed bed. The process is preferably carried out in trickle-bed reactors or in a flooded operating mode according to the fixed-bed operating mode. In order to achieve complete conversion, the hydrogenation output can undergo further reaction. This can be done by supplying the hydrogenation output to one or more reactors connected downstream that are filled with the catalyst of the invention or another catalyst. A disadvantage of this process too is that production needs to be paused when the activity of the catalyst declines and the catalyst needs to be replaced or reprocessed.

What the processes known from the prior art have in common is that they are too complex, afford poor yields and/or, as a consequence of using additives, result in products having adverse properties and/or involve pausing the process because the catalyst needs to be reprocessed from time to time. This is a particular problem when using fixed-bed catalysts.

The problem addressed by the present invention is thus that of overcoming the existing disadvantages. In particular, the problem addressed by the present invention is that of developing a process for the continuous production of methylenebis(cyclohexylamine) that combines a high level of process safety with a higher specific service life for the catalyst. The process should avoid the addition of additional external substances that contaminate high-value material streams in the high-boiling fraction.

The specific service life of the catalyst is here the amount of methylenebis(cyclohexylamine) in kg that can be produced per kg of catalyst before the catalyst needs to be replaced.

The problems addressed by the present invention are solved by the process of the invention for the continuous heterogeneous catalytic hydrogenation of MDA, in which:
a) the process is carried out in a reactor cascade comprising n serially connected reaction spaces that are each filled with catalyst and can be filled or emptied independently of one another, which are respectively given the designation Rᵢ, where 1≤i≤n, in the order in which they are connected,
b) and R₁ is temporarily disconnected from the cascade as soon as the catalyst present in R₁ has in the course of the reaction become deactivated to an undesirable degree,
   resulting in a reconfigured reactor cascade comprising i′ reaction spaces, which are given the designation R_{i′}, where 1≤i′≤(n−1), in the order in which they are connected,
   wherein each reaction space Rᵢ where 2≤i≤n becomes a reaction space Ri where 1≤i′≤(n−1),
c) the catalyst in R₁ is replaced and/or regenerated and
d) R₁ (containing the replaced and/or regenerated catalyst) is subsequently connected as reaction space R_{i′} where i′=n.

In addition, the process of the invention also has the advantage of minimizing the amount of catalyst that is replaced and/or regenerated. This conserves resources and further reduces the amount of work involved. Furthermore, the process of the invention makes it possible for the plant to be operated continuously, since the replacement of the catalyst takes place in just one reaction space and operation in the other reaction spaces is maintained, which significantly reduces plant downtime.

### Process for the Hydrogenation of MDA

The process of the invention is a process for the hydrogenation of MDA. MDA is an acronym for “methylenedianiline” and refers to a diaminodiphenylmethane-containing composition. It usually originates from the reaction of aniline and formaldehyde. The employed diaminodiphenylmethane-containing composition preferably comprises 4,4′-diaminodiphenylmethane as its principal constituent or consists thereof. Further preferably, the employed diaminodiphenylmethane-containing composition comprises 4,4′-diaminodiphenylmethane as its principal constituent and 2,4′-diaminodiphenylmethane and 2,2′-diaminodiphenylmethane as secondary constituents. However, in addition to 4,4′-diaminodiphenylmethane, 2,4′-diaminodiphenylmethane and 2,2′-diaminodiphenylmethane there may also be present further reaction products having three or more aromatic rings that are formed in the reaction of aniline and formaldehyde, in particular ones having three or more phenyl rings (“multiring compounds”).

Preference is given to using in the present process an MDA comprising at least 70% by weight of 4,4′-diaminodiphenylmethane and 0.01% to 2% by weight of N-methyl compounds (2,2′-, 2,4′- and/or 4,4′-N-methyl-methylenedianiline, in particular 2,4′- and 4,4′-N-methyl-methylenedianiline), in each case based on the total mass of compounds having aromatic rings. Even further preference is given to using an MDA consisting of 74-85% by weight of 4,4′-MDA, 3-20% by weight of 2,4′-MDA, less than 1% by weight of 2,2′-MDA and up to 1% by weight of N-methyl compounds. With the cited mixtures, a hydrogenation mixture can be particularly readily obtained that has a trans/trans content of the 4,4′ isomers of 10% to 30%.

Because of the high proportion of 4,4′-diaminodiphenylmethane in the MDA used, the methylenebis(cyclohexylamine) obtainable in the hydrogenation of the invention is mostly 4,4′-diaminodicyclohexylmethane or bis(para-aminocyclohexyl)methane. The potential presence of the corresponding 2,4′-and 2,2′-diaminophenylmethane isomers in the MDA means that 2,4′-diaminodicyclohexylmethane and 2,2′-diaminodicyclohexylmethane may likewise be present in methylenebis(cyclohexylamine). In addition, hydrogenated MDA can also contain (sometimes partially) hydrogenated multiring compounds besides methylenebis(cyclohexylamine).

The hydrogenation results in the formation of diastereoisomers. The 4,4′-diaminodicyclohexylmethane product derived from 4,4′-diaminodiphenylmethane can be in the form of trans/trans, cis/cis and cis/trans isomer and is therefore generally a mixture of these isomers in varying proportions. The process of the invention is especially well suited to the production of 4,4′-diaminodicyclohexylmethane mixtures having high trans/trans contents, particularly when using the abovementioned MDA grades.

The process of the invention for the hydrogenation of MDA is further preferably a fixed-bed process for continuous catalytic hydrogenation. Further preferably, MDA is thus hydrogenated by means of a catalytic fixed-bed process in the presence of hydrogen.

The material stream introduced per tonne of catalyst in the reactor cascade is preferably 0.1-10 t/h, further preferably 0.5-8 t/h, even further preferably 0.75-5 t/h. The material stream further preferably consists of a solution containing 5-50% by weight, further preferably 7.5-30% by weight, even further preferably 10-20% by weight, of MDA.

The hydrogen (H₂) required for the hydrogenation is preferably added stoichiometrically to marginally superstoichiometrically with respect to the desired reaction. For the process for the hydrogenation of MDA, the hydrogen (H₂) required for the hydrogenation is added in a molar ratio of 300-350 mol %, further preferably 300-330 mol %, further preferably 300-310 mol %, based on the phenyl rings present in the MDA.

The hydrogenation preferably takes place at temperatures between 50 and 200° C., preferably between 80 and 170° C., particularly preferably between 85 and 135° C. The hydrogen pressure here is preferably between 1 and 30 MPa, preferably between 5 and 15 MPa, particularly preferably between 7 and 10 MPa.

The process of the invention is thus preferably a liquid-phase hydrogenation process, i.e. a hydrogenation process carried out in the liquid phase.

A solvent can in principle be present in the hydrogenation, but does not have to be. It is however preferable to add MDA in a solvent. The proportion of the solvent is further preferably between 50% and 95% by weight, more further preferably between 70% and 92.5% by weight, even further preferably between 80% and 90% by weight, of solvent. Preferred solvents can be selected from the group consisting of primary, secondary and tertiary monohydric or polyhydric alcohols (in particular methanol, ethanol, n- and i-propanol, 1-, 2-, i- and tert-butanol, ethylene glycol and ethylene glycol mono(C1-C3)alkyl ethers), linear ethers (in particular ethylene glycol di(C1-C3)alkyl ethers), cyclic ethers (in particular tetrahydrofuran and dioxane) and alkanes (in particular n- and isoalkanes having 4-12 carbon atoms, further preferably n-pentane, n-hexane and isooctane, and cyclic alkanes, further preferably cyclohexane and decalin). Whereas alcohols can result in alkylation of the amino groups, ethers do not have this disadvantage and are thus particularly preferred. A very particularly preferred solvent is tetrahydrofuran.

However, solvent can also likewise preferably be the hydrogenation product itself.

The hydrogenation can preferably also be carried out in the presence of ammonia, a primary, secondary or tertiary amine, or a polycyclic amine having a bridging nitrogen atom.

The process of the invention is preferably a fixed-bed process for continuous catalytic hydrogenation, i.e. it is carried out in the presence of at least one heterogeneous catalyst immobilized in the fixed bed. The fixed-bed process is further preferably a process in which the reactants flow once through the reactor cascade (single-pass process).

Heterogeneous catalysts may be either unsupported catalysts or supported catalysts. In principle, both an unsupported catalyst and a supported catalyst can be used in the process of the invention.

It is possible to use just one catalyst or a mixture of catalysts. Preference is however given to using just one catalyst.

In particular, catalysts containing active metals selected from nickel, cobalt, palladium, platinum, ruthenium and/or rhodium have been found to be particularly suitable.

To increase activity, selectivity and/or service life, the catalysts may additionally comprise doping metals or comprise or have been treated with other modifiers. Preferred doping metals may be selected from the group consisting of Mo, Fe, Ag, Cr, V, Ga, In, Bi, Ti, Zr, Mn and the rare earths. Preferred modifiers are ones that allow the acid-base properties of the catalysts to be influenced, in particular alkali metals, alkaline earth metals, phosphoric acid and sulfuric acid, and compounds and salts thereof.

The catalysts can preferably be used in the form of powders or shaped bodies, for example extrudates or compressed powders. It is possible to employ unsupported catalysts, Raney-type catalysts or supported catalysts.

Preference is given to using a supported catalyst.

Preferred support materials for supported catalysts are activated carbons and inorganic oxides, particularly Al₂O₃, SiO₂, TiO₂, ZrO₂, ZnO and MgO, and also bentonites, aluminosilicates, kaolins, clays, kieselguhrs and lithium aluminates. The active metal can be applied to the support material in a manner known to those skilled in the art, for example by impregnation, spray application or precipitation. Depending on the method of catalyst production, further preparation steps known to those skilled in the art are necessary, for example drying, calcining, shaping and activation. For shaping, it is optionally possible to add further auxiliaries, for example graphite or magnesium stearate.

Preference is given to using supported catalysts containing ruthenium, rhodium or Rh/Ru combinations as essential active metals. Preferred support materials are ones based on Al₂O₃ and SiO₂.

Preference is given to using catalysts known to be employable for production of a methylenebis(cyclohexylamine) having a trans/trans content of the 4,4′-isomer of between 10% and 30% by weight, in particular between 15% and 25% by weight. Suitable catalysts are described for example in documents EP 1 366 812 A1, EP 0 066 211 A1, DE 100 54 347 A1, EP 0 392 435 A1, EP 0 630 882 A1, EP 0 639 403 A2 and U.S. Pat. No. 5,545,756 A.

Very particularly preferably, the hydrogenation is carried out in the presence of a supported catalyst that contains, applied on a support, active metal in an amount of 0.01% to 20% by weight based on the supported catalyst, and the active metal thereof is ruthenium alone or ruthenium and at least one metal of subgroups I, VII or VIII [groups 7-11] of the periodic table of the elements. This catalyst allows particularly low trans/trans contents of 4,4′-diaminodicyclohexylmethane to be achieved.

### a) Reactor Cascade

The process of the invention is carried out in a reactor cascade consisting of a plurality of reaction spaces. The reactor cascade is characterized in that 50-100 mol %, preferably 50-95 mol %, further preferably 70-90 mol %, of the hydrogen required for the hydrogenation of MDA undergoes reaction therein. In addition, it comprises n serially connected reaction spaces that are each filled with the same catalyst and can be filled or emptied independently of one another. The number n is here an integer greater than or equal to 2 that indicates how many reaction spaces that can be filled or emptied independently of one another there are. Preferably, n is a value between 2 and 10. Further preferably, n is a number selected from the range of 2 to 6; even further preferably, n=2, 3 or 4. Very particular preferably, n=2 or 3.

The individual reaction spaces are respectively given the designation Rᵢ, where 1≤i≤n, in the order in which they are connected. The expression “in the order in which they are connected” is here considered to be synonymous with “in the order of through-flow of the reactant (MDA)”. This means that the first reaction space through which the employed MDA is introduced for reaction is designated R₁, the second is designated R₂, and so on. The final reaction space is designated Rₙ. Where there are three reaction spaces, the final reaction space of the reactor cascade that is entered by the employed MDA is thus R₃.

A reaction space is in the present case understood as meaning a catalyst-filled spatial unit that is spatially separated by catalyst-free plant components from the other reaction spaces of the reactor cascade in which hydrogenation of MDA takes place at an earlier or later stage with respect to the progress of the reaction. A reaction space can thus be a subreactor that is spatially separated by catalyst-free plant components from the other subreactors of the reactor cascade in which hydrogenation takes place at an earlier or later stage with respect to the progress of the reaction. It is however also possible for the reaction space to consist of a plurality of parallel-connected subreactors in which hydrogenation of MDA takes place at the same time with respect to the progress of the reaction. Preferably, each reaction space Rᵢ consists of x parallel-connected subreactors where x=1 to 250 that are spatially separated by catalyst-free plant components from one another and from the other reaction spaces. In the case of parallel-connected subreactors, this can for example be the x parallel-connected tubes of a tube-bundle reactor. Further preferably, the number x of parallel-connected subreactors is x=2 to 250, preferably x=50 to 250, further preferably x=150 to 250.

Preferably, the catalytic hydrogenation of MDA in the reactor cascade takes place essentially isothermally, i.e. with reaction enthalpy drawn off by means of an external cooling circuit at essentially even temperature.

Preferably, the reactants flow through the reactor cascade only once, i.e. the reactor cascade is operated in single-pass mode.

Likewise preferably, the reactor cascade is a trickle-bed cascade.

### b) Removal of R₁

During the hydrogenation of MDA, the activity of the catalyst in the individual reaction spaces of the reactor cascade changes and over time declines. It was found here that the activity of a freshly introduced catalyst decreases most sharply in the first reaction space and the least in the final reaction space. R₁ is accordingly temporarily disconnected from the cascade, with the aim of replacing and/or regenerating the catalyst present therein, as soon as the catalyst present in R₁ has in the course of the reaction become deactivated to an undesirable degree.

The core of the present invention is thus that it is sufficient to replace and/or regenerate the catalyst in R₁ as soon as the catalyst present in R₁ has in the course of the reaction become deactivated to an undesirable degree,

Preferably, the point at which an undesirable degree of deactivation is reached is determined by monitoring the activity of the catalyst in R₁ while in operation and comparing this with the initial catalyst activity. Even further preferably, this is achieved by determining the normalized activity α of the catalyst present in R₁ and temporarily disconnecting R₁ from the cascade as soon as the normalized activity α of the catalyst present in R₁ falls below a defined value.

Preference is therefore given to a process for the continuous heterogeneous catalytic hydrogenation of MDA, in which:
a) the process is carried out in a reactor cascade comprising n serially connected reaction spaces that are each filled with catalyst and can be filled or emptied independently of one another, which are respectively given the designation Rᵢ, where 1≤i≤n, in the order in which they are connected,
b) the normalized activity α of the catalyst present in R₁ is determined and R₁ is temporarily disconnected from the cascade as soon as the normalized activity α of the catalyst present in R₁ falls below a defined value,
   resulting in a reconfigured reactor cascade comprising i′ reaction spaces, which are given the designation R_{i′}, where 1≤i′≤(n−1), in the order in which they are connected,
   wherein each reaction space Rᵢ where 2≤i≤n becomes a reaction space R_{i′} where 1≤i′≤(n−1),
c) the catalyst in R₁ is replaced and/or regenerated and
d) R₁ is subsequently connected as reaction space R_{i′} where i′=n.

The activity of the catalyst in R₁ is preferably monitored while in operation.

This can be done for example by continuous or occasional online monitoring of the catalyst or by withdrawing samples of the catalyst with subsequent external analysis in a laboratory-scale reference investigation at various times tₓ. The normalized activity a of the catalyst can be determined as the ratio of the conversion at time t=tₓ (dividend) and the conversion at time t=0 (divisor), the conversion being determined at a reaction temperature of 100° C., a hydrogen pressure of 80 bar and an inverse mass-related residence time of 0.330 kg (MDA)/(kg (catalyst)·h) of MDA per total mass of catalyst of all reaction spaces of the reactor cascade.

The preferred position for determining conversion in order to monitor the activity of the catalyst in R₁ is always in the same place. Further preferably, the activity of the catalyst is determined directly at the outlet of the reaction space R₁.

The actual conversion based on hydrogen consumption is calculated by evaluating the area percent values for methylenebis(cyclohexylamine) in the gas chromatogram with a factor of 1, adding 0.5 times the area percent values for the methylene(aminocyclohexyl)aniline components thereto and dividing the resulting total by the total of the area percent values for all MDA, methylene(aminocyclohexyl)aniline and methylenebis(cyclohexylamine) compounds.

As soon as the catalyst present in R₁ has in the course of the reaction become deactivated to an undesirable degree, a change is made to the connection of the individual reaction spaces. The change to the connection is preferably made as soon as the normalized activity α of the catalyst present in R₁ falls below a defined value. After the change to the connection, the catalyst in R₁ is replaced and/or regenerated.

Quantification of the degree of deactivation or the actual numerical value for the normalized activity α has little bearing on the practicability of the present invention and the achievement of the advantages of the invention. As soon as MDA conversion falls to within an undesirable range, this alone indicates that the catalyst has become deactivated or that its normalized activity α has decreased and action is required.

However, it is preferable that R₁ is disconnected from the cascade once the normalized activity α of the catalyst present in R₁ is less than 0.7, particularly preferably less than 0.5, even further preferably less than 0.3.

As soon as the catalyst present in R₁ has in the course of the reaction become deactivated to an undesirable degree, particularly when the normalized activity α of the catalyst present in R₁ falls below a defined undesirable value, a change is made to the connection of the individual reaction spaces, whereby R₁ is temporarily disconnected from the cascade. This is preferably done by redirecting the reactant and product streams. For this, the inflowing streams of unreacted MDA and hydrogen are supplied directly to reactor R₂. The streams flowing into and out of R₁ are on the other hand closed.

Disconnecting R₁ from the cascade of n reaction spaces results in a reconfigured reactor cascade comprising one reaction space fewer, i.e. (n−1) reaction spaces. This can now be redesignated on the basis of the altered configuration now present: Disconnecting R₁ from the reactor cascade comprising n reaction spaces results, from the remaining reaction spaces Rᵢ where 2≤i≤n, in a number of reaction spaces (n−1), which are given the designation R_{i′}, where 1≤i′≤(n−1), in the order in which they are connected.

### c) Replacement/Regeneration of the Catalyst

The catalyst present in R₁ is replaced and/or regenerated, i.e. the catalyst is partly or completely (preferably completely) replaced or regenerated. It is also conceivable that a portion of the catalyst is partly or completely (preferably completely) regenerated and another portion of the catalyst is partly or completely (preferably completely) replaced.

This is preferably done by releasing the pressure and emptying the residual contents of the reactor into a slop container. Any solvent still present can then be washed off the catalyst with water and the catalyst can be dried at elevated temperature in a stream of nitrogen. The catalyst can subsequently be removed and replaced with fresh catalyst.

Alternatively, the catalyst can also be regenerated. Preferred regeneration processes are described for example in CN 117654548 A, CN 110204447 B and CN 113893866 B.

After replacement and/or regeneration of the catalyst originally present in R₁, this reaction space is subsequently connected as reaction space R_{i′} where i′=n. This means that the reaction space is connected as the final reaction space in the reactor cascade. Thus, once the reaction space formerly designated R₁ has been connected as new reaction space Rₙ, the reactor cascade again comprises n serially connected reaction spaces that are each filled with catalyst and can be filled or emptied independently of one another. The reconfigured reactor cascade comprising n reaction spaces can of course undergo the process of the invention again as soon as the catalyst now present in R₁ is no longer sufficiently active.

The present invention is based on a reactor concept in which the reactor consists of two or more reaction spaces arranged in series with respect to one another. Such an arrangement allows the individual reaction spaces to be replaced without reprocessing or replacing the portion of the catalyst that is still active.

During replacement or regeneration of the catalyst, the hydrogenation can in principle be halted or continued. Operation of the hydrogenation is preferably continued during the replacement or regeneration, since this is better for the space-time yield of the plant. Further preferably, the operation of the hydrogenation is continued with reduced load, since this allows particularly good results to be achieved.

In a preferred embodiment of the process n=2, i.e. the reactor cascade consists of two reaction spaces R₁ and R₂ that can be filled or emptied independently of one another and are respectively given the designation R₁ and R₂ in the order in which they are connected. R₁ is temporarily disconnected from the cascade as soon as the catalyst present in R₁ has in the course of the reaction become deactivated to an undesirable degree. The reaction is thus carried out only in R₂, and R₂ is from then on designated reaction space R_{1′}. The catalyst in R₁ is replaced and/or regenerated. R₁ is subsequently connected as reaction space R₂.

Further preferably, the point at which an undesirable degree of deactivation is reached is determined by monitoring the activity of the catalyst in R₁ while in operation and comparing this with the initial catalyst activity. Even further preferably, the normalized activity α of the catalyst present in R₁ is determined and R₁ is temporarily disconnected from the cascade as soon as the normalized activity α of the catalyst present in R₁ falls below a defined value.

The continuous hydrogenation of the invention can only be carried out in the abovementioned reactor cascade. It is however preferable that the reaction in the reactor cascade is followed by a reaction in a finisher (which can optionally comprise two or more sub-finishers). In said unit, preferably in the presence of at least one different catalyst, the conversion for the hydrogenation of MDA is increased further. This finisher (downstream reactor) is not part of the reactor cascade.

The material stream introduced into the finisher per tonne of catalyst in the reactor cascade is preferably 0.15-25 t/h, further preferably 0.75-20 t/h, even further preferably 1-15 t/h.

Particularly good results can be achieved when the reaction is carried out essentially isothermally (i.e. with reaction enthalpy drawn off by means of an external cooling circuit at essentially constant temperature) in the reactor cascade and essentially adiabatically (i.e. without cooling and with little heat loss) in the finisher.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 24191090, filed Jul. 26, 2025, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for the continuous heterogeneous catalytic hydrogenation of MDA, characterized in that
a) the process is carried out in a reactor cascade comprising n serially connected reaction spaces that are each filled with catalyst and can be filled or emptied independently of one another, which are respectively given the designation Rᵢ, where 1≤i≤n, in the order in which they are connected,
b) and R₁ is temporarily disconnected from the cascade as soon as the catalyst present in R₁ has in the course of the reaction become deactivated to an undesirable degree,
resulting in a reconfigured reactor cascade comprising i′ reaction spaces, which are given the designation R_{i′}, where 1≤i′≤(n−1), in the order in which they are connected,
wherein each reaction space Rᵢ where 2≤i≤n becomes a reaction space R_{i′} where 1≤i′≤(n−1),
c) the catalyst in R₁ is replaced and/or regenerated and
d) R₁ is subsequently connected as reaction space R_{i′} where i′=n.

2. Process according to claim 1,
characterized in that
the MDA used comprises at least 70% by weight of 4,4′-diaminodiphenylmethane and 0.01% to 2% by weight of N-methyl compounds, in each case based on the total mass of compounds having aromatic rings.

3. Process according to claim 1,
characterized in that
the process is a fixed-bed process for continuous catalytic hydrogenation.

4. Process according to claim 1,
characterized in that
the catalyst is a supported catalyst.

5. Process according to claim 4,
characterized in that
the catalyst
contains, applied on a support, active metal in an amount of 0.01% to 20% by weight based on the supported catalyst, and
the active metal thereof is ruthenium alone or ruthenium and at least one metal of subgroups I, VII or VIII [groups 7-11] of the periodic table of the elements.

6. Process according to claim 1,
characterized in that
each reaction space Rᵢ consists of x parallel-connected subreactors where x=1 to 250 that are spatially separated by catalyst-free plant components from one another and from the other reaction spaces.

7. Process according to claim lany of the preceding claims, characterized in that the catalytic hydrogenation of MDA in the reactor cascade takes place essentially isothermally.

8. Process according to claim 1,
characterized in that
the point at which an undesirable degree of deactivation is reached is determined by monitoring the activity of the catalyst in R₁ while in operation and comparing this with the initial catalyst activity.

9. Process according to claim 8,
characterized in that
the normalized activity α of the catalyst present in R₁ is determined and R₁ is temporarily disconnected from the cascade as soon as the normalized activity α of the catalyst present in R₁ falls below a defined value.

10. Process according to claim 9,
characterized in that
the normalized activity α of the catalyst present in R₁ is less than 0.7.

11. Process according to claim 1,
characterized in that
the hydrogenation is continued during the replacement or regeneration of the catalyst.

12. Process according to claim 1,
characterized in that
a) the process is carried out in a reactor cascade comprising two serially connected reaction spaces that are each filled with catalyst and can be filled or emptied independently of one another, which are respectively given the designation R₁ and R₂ in the order in which they are connected,
b) and R₁ is temporarily disconnected from the cascade as soon as the catalyst present in R₁ has in the course of the reaction become deactivated to an undesirable degree,
so that the reaction is carried out only in R₂, wherein R₂ is from then on designated reaction space R_{1′},
c) the catalyst in R₁ is replaced and/or regenerated and
R₁ is subsequently connected as reaction space R_{2′}.

13. Process according to claim 1,
characterized in that
the reaction in the reactor cascade is followed by a reaction in a finisher.

14. Process according to claim 13,
characterized in that
the reaction is carried out essentially isothermally in the reactor cascade and essentially adiabatically in the finisher.
